(19) United States
(12) Patent Application Publication
(10) Pub. No.: US 2023/0373821 A1
(43) Pub. Date: Nov. 23, 2023

(54) UV LED ASSEMBLY AND WATER PURIFIER EQUIPPED WITH SAME

(71) Applicant: Kyung Ah SEO, Paju-si (KR)

(72) Inventor: Kyung Ah SEO, Paju-si (KR)

(73) Assignee: B.L.I Inc., Paju-si (KR)

(21) Appl. No.: 17/836,960

(22) Filed: Jun. 9, 2022

(30) Foreign Application Priority Data
May 20, 2022 (KR) 10-2022-0062022
May 20, 2022 (KR) 10-2022-0062034

(51) Int. Cl. C02F 1/32; A61L 2/26; A61L 2/10

(52) U.S. Cl. CPC C02F 1/325; A61L 2/26; A61L 2/10; C02F 2303/04; C02F 2201/3222; A61L 2202/11

(57) ABSTRACT

Disclosed is an UV LED assembly and a water purifier capable of killing harmful bacteria existed in a water tank. The water purifier includes a water purifier including a through-hole formed through a side wall of a water tank such that the inside and outside of the water tank communicate with each other; a body pipe inserted in the through-hole to be installed inside and outside the water tank, in which the end of the body pipe inside the water tank may be closed; and a stop nut seating the UV LED sub-assembly in the hollow section by being thread-fastened to a side of the end of the body pipe disposed outside the water tank after a body of a UV LED sub-assembly, which is integrated with urethane, is inserted and fitted in a hollow section of the body pipe from the outside of the water tank.

### BACKGROUND OF THE INVENTION

### Field of the Invention

The present disclosure relates to a water purifier. More specifically, the present disclosure relates to a technique of installing a UV lamp, which can emit infrared light to water in a water purifier, in a water purifier to be able to kill harmful bacteria that may exist in the water kept in the water purifier.

### Description of the Related Art

Conventionally, there has been a water purifier that purifies a water stored in the water purifier by means of UV LED.

Specifically, as disclosed in FIG. 10 of the prior art, UV LED sterilization units (124) and (134) are provided in a temporary water tank (120) and a main water tank (130) of the water purifier, respectively.

However, the UV LED sterilization units (124) and (134) embedded in the water of the temporary water tank (120) and the main water tank (130) frequently fail due to a penetration of moisture or an abnormal surge current and so on.

Accordingly, in order to replace the UV LED sterilization units (124) and (134), since the water tanks (120) and (130), in which the UV LED sterilization units (124) and (134) are built, are cut and the accessory assembly device for connecting the UV LED sterilization units (124) and (134) with the water storage tanks (120) and (130) must also be disassembled or cut, the maintenance process thereof is very complicated.

Therefore, in the prior art, when the failure of the UV LED sterilization units (124) and (134) occurs, the entire water tank having the UV LED sterilization units (124) and (134) is replaced in many cases. Accordingly, there is a problem in that the maintenance cost of the water purifier increases.

### PATENT LITERATURE

Patent Literature 1: Korean Patent Publication No. 10-2009-0069548 (published on Jul. 1, 2009)

### SUMMARY OF THE INVENTION

In order to solve the problems of the related art described above, the present disclosure makes it possible to very simply replace a ‘UV LED sub-assembly’ equipped with an UV LED lamp, which frequently fails, from the outside of a water tank even without disassembling or opening the water tank.

Accordingly, a user or a manager who check a lamp, etc., which show failure of a ‘UV LED sub-assembly’ equipped with a UV LED lamp while using a water purifier may turn and open a stop nut outside a water tank of the water purifier, remove the broken UV LED sub-assembly, put in a new UV LED sub-assembly, and then turn and close the stop nut, whereby replacement is finished.

Since it is possible to replace a UV LED sub-assembly in this very simple method, the water purifier to which the present disclosure is applied makes it possible to reduce maintenance time and remove the necessity for discarding a water purifier itself with a broken sterilizer due to difficulty in maintenance, so the lifespan of the water purifier increases.

According to an aspect of the invention to achieve the object described above, there is provided a water purifier including: a through-hole (52) formed through a side wall of a water tank (51) such that the inside and outside of the water tank communicate with each other; a body pipe (1) inserted in the through-hole (52) to be installed inside and outside the water tank, in which the end of the body pipe (1) inside the water tank may be closed; and a stop nut (10) seating the UV LED sub-assembly (41) in the hollow section (100) by being thread-fastened to a side of the end of the body pipe (1) disposed outside the water tank after a body (6-1) of a UV LED sub-assembly (41), which is integrated with urethane, is inserted and fitted in a hollow section (100) of the body pipe (1) from the outside of the water tank.

In addition, as for the shape of the body pipe (1), a body pipe separation plate (32) having a constant thickness is perpendicularly integrated with the body pipe (1) along the outer circumference of the body pipe (1) at a predetermined distance from an end of the body pipe (1), and a first spiral pipe (33) and a second spiral pipe (34) are formed at a side and the other side, respectively, in the axial direction of the body pipe (1) with reference to the body pipe separation plate (32) fixed to the body pipe.

Meanwhile, a body pipe packing (8) is fitted on the first spiral pipe (33) of the body pipe (1) and an end of the first spiral pipe (33) is inserted in the through-hole (52) formed through a side wall of the water tank from the inside of the water tank, whereby a body pipe stop nut (9) outside the water tank (51) is firmly coupled to the first spiral pipe (33).

In addition, an O-ring (12) is fitted on the second spiral pipe (34) of the body pipe (1) and then the outer circumference of a quartz glass (2) is fitted in an inner groove (3-1) of a glass packing (3), and the quartz glass (2) with the glass packing (3) fitted on the outer circumference thereof is brought into close contact with the hollow section of the glass stop nut and then the hollow section is sealed, whereby the glass stop nut (4) is firmly coupled to the second spiral pipe (34).

Meanwhile, the UV LED sub-assembly (41) is a combination of a stop nut packing (11), a PCB stop nut (7), a PCB assembly (5), and a PCB holder (6), and the PCB stop nut (7) and the PCB assembly (5) are inserted together in a hole of the PCB holder (6) to be integrated, and the stop nut packing (11) is coupled to the lower portion of the PCB stop nut (7).

In addition, an O-ring (13) is further disposed on the front of the UV LED sub-assembly (41) to firmly seat the UV LED sub-assembly (41) in the hollow section (100) of the body pipe (1).

Meanwhile, the UV LED lamp (5-1) is mounted on the front end of the PCB assembly (5), and the infrared light emitted from the UV LED lamp (5-1) passes through the transparent quartz glass (2) and sterilizes bacteria that may exist in the water in the water tank.

In addition, the UV LED sub-assembly that is a combination of a stop nut packing (11), a PCB stop nut (7), a PCB assembly (5), and a PCB holder (6), wherein the PCB stop nut (7) and the PCB assembly (5) are inserted together in a hole of the PCB holder (6), so the PCB stop nut (7), the PCB assembly (5), and the PCB holder (6) are integrated, and the stop nut packing (11) is coupled to the lower portion of the PCB stop nut (7).

### BRIEF DESCRIPTION OF THE DRAWINGS

The above and other objects, features and advantages of the present invention will be more apparent from the following detailed description taken in conjunction with the accompanying drawings, in which:

FIG. 1A is an exploded front view of an UV LED assembly of the present invention separated from a water tank;

FIG. 1B is an exploded perspective view of an UV LED assembly of the present invention separated from a water tank;

FIG. 2A is a cross-sectional view illustrating a structure that an UV LED assembly of the present invention is assembled in a water tank.

FIG. 2B is a perspective view illustrating a shape of an UV LED assembly of the present invention separated from a water tank;

FIG. 3 is a perspective view of the body pipe;

FIG. 4 is a cross-sectional view illustrating a shape of a body pipe cut in the axial direction;

FIG. 5 is a front view, a cross-sectional view, and a perspective view of a glass packing;

FIG. 6A is a front view illustrating a shape in which an UV LED lamp is mounted on the front end of a PCB assembly;

FIG. 6B is a cross-sectional view illustrating a shape in which an UV LED lamp is mounted on the front end of a PCB assembly;

FIG. 7 is a front view of an UV LED lamp mounted on the front end of a PCB assembly, illustrating a shape mounted on the water tank, and looking at the front end of a PCB assembly from the inside of a water tank.

FIG. 8 is a side view of a structure in which an UV LED assembly of the present invention is mounted on a water tank as viewed from the side thereof;

FIG. 9 is a schematic diagram illustrating a replacement of an UV LED sub-assembly; and

FIG. 10 is a schematic diagram illustrating the prior art of the present invention.

### REFERENCE SIGNS LIST

1: body pipe
2: quartz glass
3: glass packing
3-1: inner groove of glass packing
4: glass stop nut
5: PCB assembly
6: PCB holder
6-1: body
7: PCB stop nut
8: body pipe packing
9: body pipe stop nut
10: stop nut
11: stop nut packing
12, 13: O-rings
32: body pipe separation plate
33: first spiral pipe
34: second spiral pipe
41: UV LED sub-assembly
51: water tank
52: through-hole of water tank
100: hollow section of body pipe

### DETAILED DESCRIPTION OF THE PREFERRED EMBODIMENT

Hereafter, the function, configuration, and operation of a water purifier according to the present invention are described in detail with reference to the accompanying drawings.

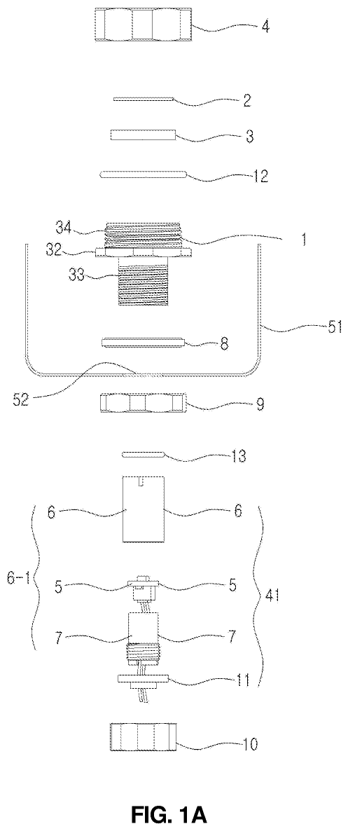

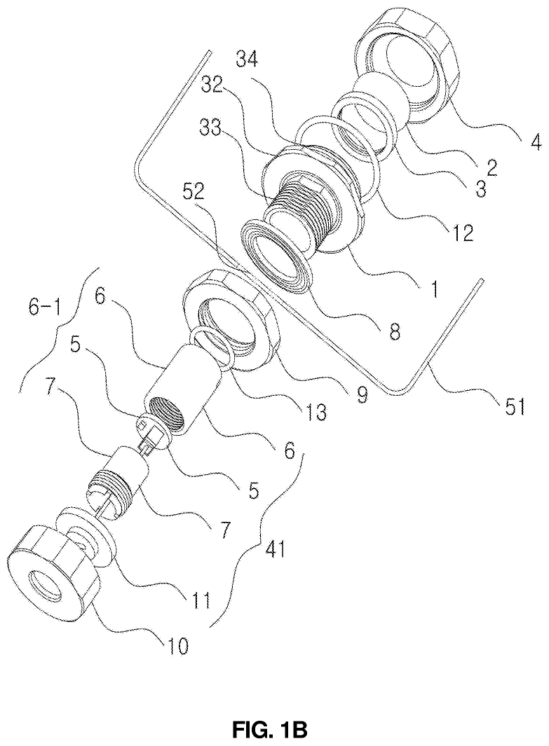

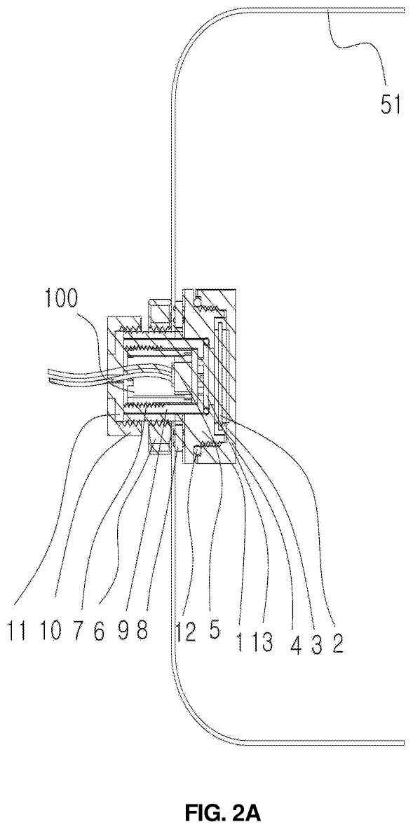

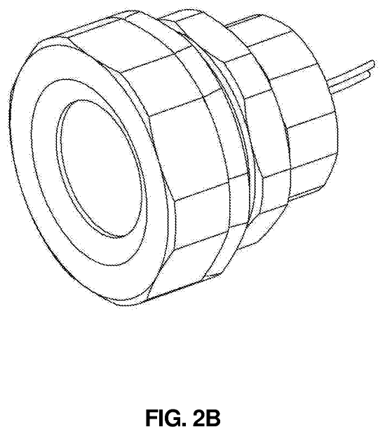

As shown in FIGS. 1A, 1B, 2A, and 2B, the present disclosure is a UV LED assembly for sterilizing the inside of a water tank of a water purifier including:

a through-hole (52) formed through a side wall of a water tank (51) such that the inside and outside of the water tank communicate with each other;

a body pipe (1) inserted in the through-hole (52) to be installed inside and outside the water tank, in which the end of the body pipe (1) inside the water tank may be closed; and

a stop nut (10) seating the UV LED sub-assembly (41) in the hollow section (100) by being thread-fastened to a side of the end of the body pipe (1) disposed outside the water tank after a body (6-1) of a UV LED sub-assembly (41) is inserted and fitted in a hollow section (100) of the body pipe (1) from the outside of the water tank.

In detail, the end of the body pipe (1) inside the water tank is sealed by a packing, an O-ring, a nut, a quartz glass, or the like, so the body pipe is made watertight with minimum permeation of moisture.

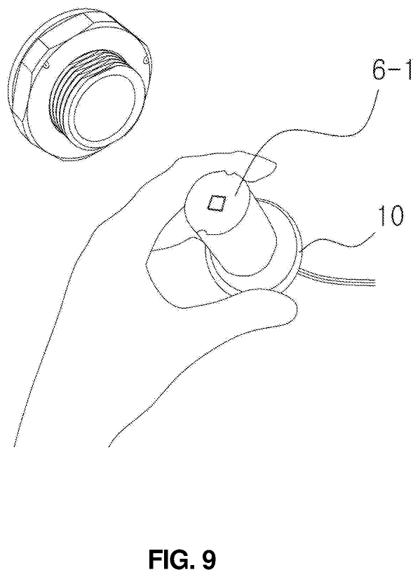

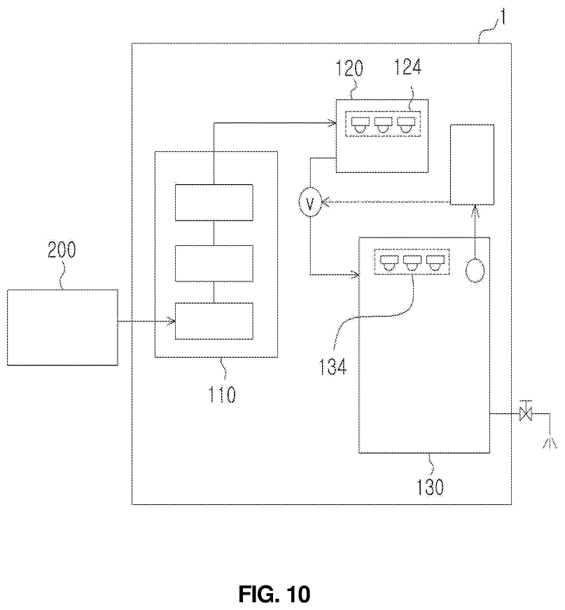

Next, the definition and the organic configuration of the ‘LED sub-assembly’ are described with reference to FIGS. 1B and 9.

The ‘UV LED sub-assembly (41)’ is a combination of a stop nut packing (11), a PCB stop nut (7), a PCB assembly (5), and a PCB holder (6).

The PCB stop nut (7) and the PCB assembly (5) are inserted together in a hole of the PCB holder (6) to be integrated, and the stop nut packing (11) is coupled to the lower portion of the PCB stop nut (7).

The inside of the PCB holder (6) is empty as in the shape shown in FIG. 2A.

The PCB stop nut (7), the PCB assembly (5), and the PCB holder (6) are integrated with urethane not to be separated, which is defined as the ‘body (6-1) of the UV LED sub-assembly (41)’ (see FIG. 9).

That is, the assembly of the body (6-1) of the UV LED sub-assembly (41) with the addition of the stop nut packing (11) is defined as the ‘UV LED sub-assembly (41)’ (see FIG. 1b).

The body (6-1) of the UV LED sub-assembly (41) that can be inserted in a body hollow section (100) is shown in FIG. 9.

Meanwhile, when a UV LED lamp (5-1) seated on the front of the PCB assembly (5) of the water purifier fails, a manager or a user of the water purifier turns and opens the stop nut (10) by hand, removes the failed UV LED sub-assembly (41) behind the stop nut 10, mounts a new UV LED sub-assembly (41) at the place, and then turns and couples the stop nut (10) to a first spiral pipe (33), thereby finishing the replacement and maintenance of the UV LED assembly for sterilizing the inside of the water tank.

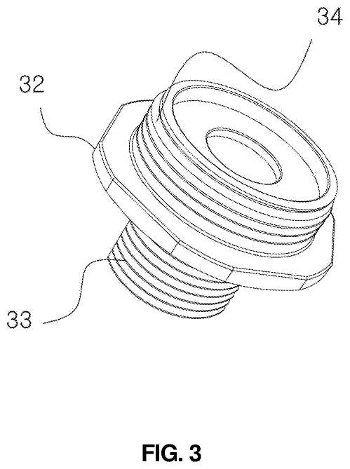

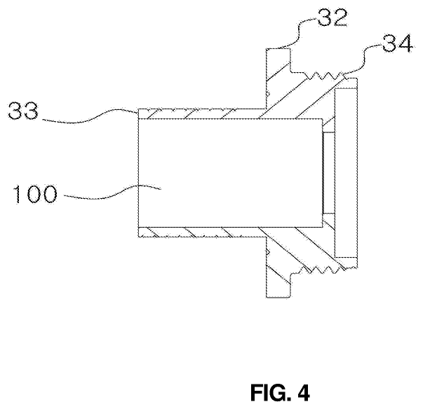

Next, the shape of the body pipe (1) shown in FIGS. 1, 3, and 4 is described.

The body pipe is installed through a side wall of the water tank while connecting the inside and outside of the water tank.

As for the shape of the body pipe (1), a body pipe separation plate (32) having a constant thickness is perpendicularly integrated with the body pipe (1) along the outer circumference of the body pipe (1) at a predetermined distance from an end of the body pipe (1).

The body pipe separation plate (32) should be designed not to pass through the through-hole (52) formed through the wall of the water tank when the body pipe (1) is inserted and fixed through the wall of the water tank, so the diameter of the body pipe separation plate (32) should be larger by a predetermined size than the through-hole (52).

Further, a first spiral pipe (33) and a second spiral pipe (34) are formed at a side and the other side, respectively, in the axial direction of the body pipe (1) with reference to the body pipe separation plate (32) fixed to the body pipe (1).

Next, the structure in which the body pipe (1) is fixed to a side wall of the body pipe (1) is described with reference to FIGS. 1 to 4.

A body pipe packing (8) is fitted on the first spiral pipe (33) of the body pipe (1), an end of the first spiral pipe (33) is inserted in a through-hole formed through a side wall of the water tank from the inside of the water tank (51), and a body pipe stop nut (9) outside the water tank (51) is turned to be firmly coupled to the first spiral pipe (33).

As described above, since the body pipe packing (8) is pressed between the body pipe separation plate (32) and the body pipe stop nut (9) with the body pipe packing (8), which is made of silicone, in close contact with the wall of the water tank, the body pipe (1) is very hermetically and firmly coupled to the through-hole (52) of the water tank.

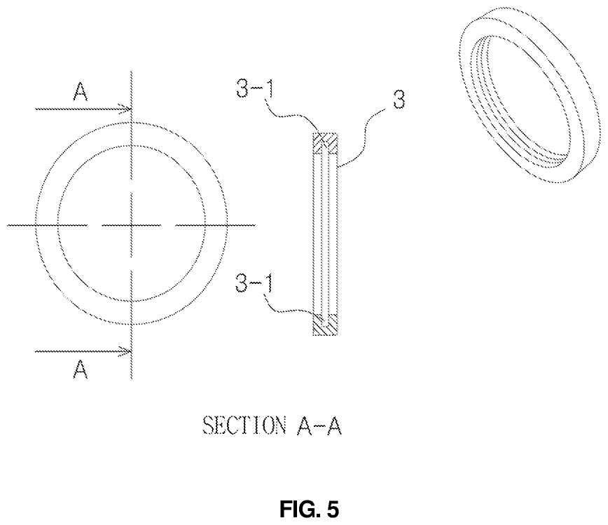

Next, the coupling relationship of other components of the present disclosure with the second spiral pipe (34) is described with reference to FIGS. 1 to 5.

An O-ring (12) is fitted on the second spiral pipe (34) of the body pipe (1) and then the outer circumference of the quartz glass (2) is fitted in an inner groove (3-1) of a glass packing (3).

The transparent quartz glass (2) with the glass packing (3) fitted on the outer circumference thereof is brought into close contact with the hollow section of a glass stop nut (4) and then the hollow section is sealed, whereby the glass stop nut (4) is firmly coupled to the second spiral pipe (34).

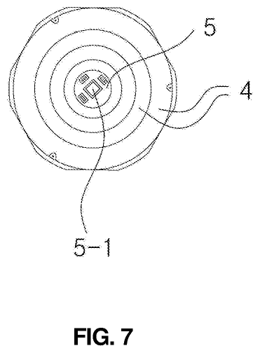

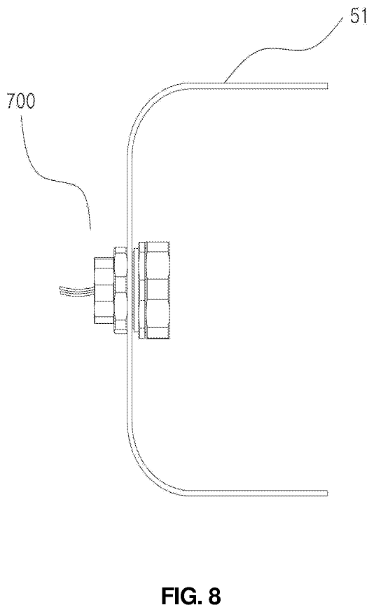

As shown in FIG. 7, infrared light emitted from the UV LED lamp (5-1) passes through the transparent quartz glass (2) and sterilizes the water kept in the water tank.

Accordingly, the transparent quartz glass (2) should be transparent without contaminants and should have no scratch. Further, the UV transmittance of the transparent quartz glass (2) should be 90% or more.

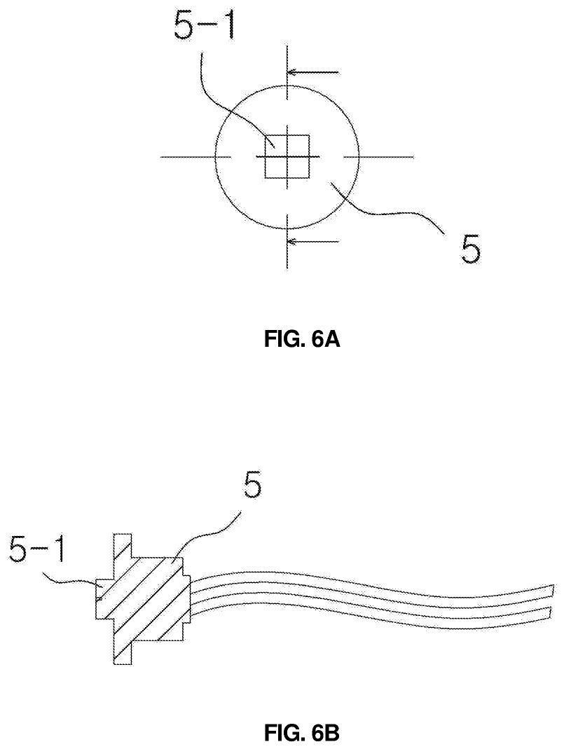

Next, the definition and the organic configuration of the ‘LED sub-assembly (41)’ are described with reference to FIGS. 1, 2, 6, and 7.

The ‘UV LED sub-assembly (41)’ is composed of a stop nut packing (11), a PCB stop nut (7), a PCB assembly (5), and a PCB holder (6).

The PCB stop nut (7), the PCB assembly (5), and the PCB holder (6) are integrated with urethane not to be separated, which is defined as the ‘body (6-1) of the UV LED sub-assembly (41)’ (see FIG. 9).

Meanwhile, an O-ring (13) is further disposed on the front of the UV LED sub-assembly (41) to firmly seat the UV LED sub-assembly (41) in the hollow section (100) of the body pipe (1), whereby hermeticity of the UV LED assembly can be improved.

As for the term ‘front’ used in the present disclosure, the direction from the UV LED sub-assembly (41) to the water tank (51) in FIG. 1B is defined as ‘front’, and the opposite direction is defined as ‘rear’.

As shown in FIG. 7, the UV LED lamp (5-1) is mounted on the front end of the PCB assembly (5), and the infrared light emitted from the UV LED lamp (5-1) passes through the transparent quartz glass (2) and sterilizes bacteria that may exist in the water in the water tank.

Next, main components of the present disclosure are made of the flowing materials.

The body pipe (1), the glass stop nut (4), the body pipe stop nut (9), and the stop nut (10) are made of polyphenylene sulfide (PPS).

PPS is thermoplastic resin having excellent mechanical properties such as strength, heat resistance, electrical insulation, and dimensional stability.

The glass packing (3), the O-rings (12 and 13), the body pipe packing (8), and the stop nut packing (11) are made of silicone.

Meanwhile, the PCB holder (6) and the PCB stop nut (7) are made of polyoxymethylene.

The present disclosure makes it possible to very simply replace a ‘UV LED sub-assembly’ equipped with an UV LED lamp, which frequently fails, from the outside of a water tank even without disassembling or opening the water tank

Accordingly, maintenance time can be dramatically reduced, and the necessity for discarding a water purifier itself with a broken sterilizer due to difficulty in maintenance is removed, thereby increasing the lifespan of the water purifier.

In addition, multiple O-rings and packings are applied to the UV LED assembly of the present disclosure, thereby preventing permeation of water or moisture and minimizing failure of UV LED lamps.

While the present invention has been described with respect to the specific embodiments, it will be apparent to those skilled in the art that various changes and modifications may be made without departing from the spirit and scope of the invention as defined in the following claims.

What is claimed is:

1. A water purifier comprising:
a through-hole (52) formed through a side wall of a water tank (51) such that the inside and outside of the water tank communicate with each other;
a body pipe (1) inserted in the through-hole (52) to be installed inside and outside the water tank, in which the end of the body pipe (1) inside the water tank may be closed; and
a stop nut (10) seating the UV LED sub-assembly (41) in the hollow section (100) by being thread-fastened to a side of the end of the body pipe (1) disposed outside the water tank after a body (6-1) of a UV LED sub-assembly (41), which is integrated with urethane, is inserted and fitted in a hollow section (100) of the body pipe (1) from the outside of the water tank.

2. The water purifier of claim 1, wherein as for the shape of the body pipe (1), a body pipe separation plate (32) having a constant thickness is perpendicularly integrated with the body pipe (1) along the outer circumference of the body pipe (1) at a predetermined distance from an end of the body pipe (1), and
a first spiral pipe (33) and a second spiral pipe (34) are formed at a side and the other side, respectively, in the axial direction of the body pipe (1) with reference to the body pipe separation plate (32) fixed to the body pipe.

3. The water purifier of claim 2, wherein a body pipe packing (8) is fitted on the first spiral pipe (33) of the body pipe (1) and an end of the first spiral pipe (33) is inserted in the through-hole (52) formed through a side wall of the water tank from the inside of the water tank, whereby a body pipe stop nut (9) outside the water tank (51) is firmly coupled to the first spiral pipe (33).

4. The water purifier of claim 3, wherein an O-ring (12) is fitted on the second spiral pipe (34) of the body pipe (1) and then the outer circumference of a quartz glass (2) is fitted in an inner groove (3-1) of a glass packing (3), and
the quartz glass (2) with the glass packing (3) fitted on the outer circumference thereof is brought into close contact with the hollow section of the glass stop nut and then the hollow section is sealed, whereby the glass stop nut (4) is firmly coupled to the second spiral pipe (34).

5. The water purifier of claim 4, wherein the UV LED sub-assembly (41) is a combination of a stop nut packing (11), a PCB stop nut (7), a PCB assembly (5), and a PCB holder (6), and
the PCB stop nut (7) and the PCB assembly (5) are inserted together in a hole of the PCB holder (6) to be integrated, and the stop nut packing (11) is coupled to the lower portion of the PCB stop nut (7).

6. The water purifier of claim 5, wherein an O-ring (13) is further disposed on the front of the UV LED sub-assembly (41) to firmly seat the UV LED sub-assembly (41) in the hollow section (100) of the body pipe (1).

7. The water purifier of claim 6, wherein the UV LED lamp (5-1) is mounted on the front end of the PCB assembly (5), and the infrared light emitted from the UV LED lamp (5-1) passes through the transparent quartz glass (2) and sterilizes bacteria that may exist in the water in the water tank.

8. A UV LED sub-assembly that is a combination of a stop nut packing (11), a PCB stop nut (7), a PCB assembly (5), and a PCB holder (6),
wherein the PCB stop nut (7) and the PCB assembly (5) are inserted together in a hole of the PCB holder (6), so the PCB stop nut (7), the PCB assembly (5), and the PCB holder (6) are integrated, and the stop nut packing (11) is coupled to the lower portion of the PCB stop nut (7).
